(19) United States
(12) Patent Application Publication
(10) Pub. No.: US 2021/0267253 A1
(43) Pub. Date: Sep. 2, 2021

(54) COMPOSITIONS OF LIPASES AND PREGASTRIC ESTERASES FOR MAMMALIA NUTRITION SUPPORT

(71) Applicant: ATR THRIVE, LLC, Saint Louis, MO (US)

(72) Inventors: Josephine GAUL, Benton, MO (US); Neville MCNAUGHTON, Saint Louis, MO (US)

(73) Assignee: ATR THRIVE, LLC, Saint Louis, MO (US)

(21) Appl. No.: 17/262,299

(22) PCT Filed: Jul. 23, 2019
(86) PCT No.: PCT/US19/43054

§ 371 Date: Jan. 22, 2021

Related U.S. Application Data
(60) Provisional application No. 62/702,442, filed on Jul. 24, 2018.
(60) Provisional application No. 62/821,604, filed on Mar. 21, 2019.

(51) Int. Cl. A23L 33/00; A23L 29/00; A23L 33/115; A23L 33/135; A23L 33/125

(52) U.S. Cl. CPC A23L 33/40; A23L 29/06; A23L 33/125; A23L 33/135; A23L 33/115

(57) ABSTRACT

Compositions and methods are disclosed containing lingual lipase or another pregastric esterase as a nutritional composition for use in neonatal mammals or mammals with fat maldigestion. The compositions may be used as a stand-alone feed, or an additive to milk, milk or replacers. The composition may also include micronutrients, macronutrients, and bioactive dietary components for neonate mammals or mammals of any age with fat maldigestion Mammals include ruminants, porcines, horses, camelids, dogs, cats, or humans. Lingual lipase or pregastric esterase as a nutritional supplement may substantially improve the digestion of fats in mammals unable to effectively digest fats. The lingual lipase or esterase is preferably from an animal source. The composition may contain butterfat, micellar casein, whey, non-denatured proteins, and a source of lactose. Methods of treating fat maldigestion with the compositions are also disclosed.

### CROSS REFERENCE TO RELATED APPLICATIONS

This application claims the benefit of U.S. patent application 62/702,442, filed Jul. 24, 2018, and U.S. patent application 62/821,604 filed Mar. 21, 2019.

### FIELD OF THE INVENTION

This disclosure pertains to compositions and methods containing lipases, including lingual and pregastric esterases, for feeding mammals experiencing failure to thrive, medical conditions related to fat maldigestion, nutritional stress, premature birth, and post-surgical recovery conditions. The compositions are particularly useful in neonates not naturally fed at the breast or udder and more mature mammals with fat maldigestion.

### BACKGROUND

The digestion of fats by mammals requires any of several types of lipase enzymes. The inability to digest fats can lead to malnutrition, and in certain populations, this may be a serious problem.

In the case of newborn mammal infants (neonates), fat digestion is critical yet can be a physiological challenge in cases where a newborn does not, for whatever reason, suckle natural milk at the breast or udder of the mother. All mammalian infants must begin to digest fats immediately after birth, but without natural suckling, lipases required for fat digestion may not be available in sufficient quantity, and fat digestion is much less efficient. Deficiencies in early fat digestion can cause neonatal malnutrition and failure-to-thrive. Free fatty acids from the digestion of fats are both nutrition, and defining elements of the stomach, being selective, protective and inhibitory to unwanted organisms, thereby being a defining element in the establishment of the gut microbiome. This can be a significant societal problem for humans, and a major economic problem for livestock.

A related issue are humans or mammals with other fat maldigestion conditions, such as cystic fibrosis, pancreatic lipase insufficiency, nonalcoholic fatty liver, alcoholic fatty liver or post-surgical conditions.

In the case of neonatal mammals, as the neonate (human or animal) transitions from the womb to the outside world, the digestive tract must activate to provide sustenance. Several things happen at this point. The neonate must transition from high carbohydrate nutrition in the womb to getting much of its nutrition through the digestive tract in the form of fats. The newborn has no digestive tract bacteria that are critical in all animals to aid digestion, and in most species the newborn digestive tract is small and immature for a few days at least.

The first milk produced by the mother after birth is colostrum, which delivers its nutrients in a very concentrated low-volume form. In most species colostrum contains antibodies to protect the newborn from disease and in most species has a higher protein content than ordinary milk. The fat content of colostrum compared to ordinary milk varies by species, being higher in some species, and lower in other species.

The term “fats” refers primarily to triacylglycerols, also termed triglycerides, or triglyceride esters, which comprise three fatty acids linked to glycerol with ester linkages. Other fats include phospholipids and cholesterol. This invention is concerned with triacylglycerol fats only.

The digestion of triacylglycerol fats requires the action of lipase or esterase enzymes that cleave the ester linkages to form partial glycerides and free fatty acids (FFA's). This is necessary because fats are hydrophobic and are not effectively soluble in the intestine and do not effectively cross the digestive mucosa.¹ Triacylglycerol fats must be broken down into FFA's to cross the digestive mucosa. Once absorbed in the intestinal mucosa, FFA's are bound to fatty-acid binding protein and reesterified with the aid of acetyl-CoA to triacylglycerols again.² The re-formed triacylglycerols are assembled into chylomicrons in the endoplasmic reticulum in the absorptive cells (enterocytes) of the small intestine.³ Chylomicrons transport dietary lipids from the intestine to other locations in the body. ¹ Margit Hamosh, A Review. Fat Digestion in the Newborn: Role of Lingual Lipase and Preduodenal Digestion, Pediatric Research, 13, 615-622 (1979), doi: 10.1203/00006450-197905000-00008² Id.³ Mahmood Hussain, M “A proposed model for the assembly of chylomicrons,” Atherosclerosis, 148(1), 1-15 (2000), doi: 10.1016/S0021-9150(99)00397-4

Several lipases with varying chemistry play a role in digestion. A principal lipase in more mature mammals is pancreatic lipase. With regards to newborns, lipases are present in milk⁴ and pregastric lipases are formed during suckling.⁵ Bile salts are required for both pancreatic and milk lipase.⁶ ⁴ Lipases in bovine milk: C. V. Patel et al., “Bovine Milk Lipase. II. Characterization” J.Dairy Sci.1968, 51(12), 1879-1886 https://doi.org/10.3168/jds.S0022-0302(68)87306-0; lipases in human milk: William G Manson, Lawrence T Weaver, “Fat digestion in the neonate,” Arch. Dis. Childhood, 1997, 76, F206-F211; PMID: 9175955.⁵ Nelson, J. H., et al., “Pregastric Esterase and other Oral Lipases—A Review,” J. Dairy Sci. 1977, 60(3) 327-362, doi: 10.3168/jds.S0022-0302(77)83873-3⁶ Manson 1997, n. 4

This invention is principally directed to various preduodenal lipases, in particular lingual lipase, pregastric lipase, and gastric lipase.⁷ Esterases are closely related enzymes that may also be of value in this invention.⁸ ⁷Hamosh, M; and Hamosh, P; “Gastric Lipase: Characteristics and Biological Function of Preduodenal Digestive Lipases” in “Esterases, Lipases, and Phospholipases: From Structure to Clinical Significance Volume 266 of Nato Science Series A” Editors: M. I. Mackness, M. Clerc; Springer 1994, pp. 139-148. ISBN 1489909931, 9781489909930; Hernell, O; Blackberg L, “Molecular aspects of fat digestion in the newborn,” Acta Paediatr Suppl 1994, 405, 65-9; https://doi.org/10.1111/j.1651-2227.1994.tb13401.x; Roussel, Alain, et al., “Crystal Structure of Human Gastric Lipase and Model of Lysosomal Acid Lipase, Two Lipolytic Enzymes of Medical Interest,” J. Biol. Chem. 1999, 274(24), 16995-17002; https://doi.org/10.1074/jbc.274.24.16995⁸Fojan, P. et al., “What distinguishes an esterase from a lipase: A novel structural approach,” Biochimie, 2000, 82 (11) 1033-1041, https://doi.org/10.1016/50300-9084(00)01188-3

Lingual lipase is produced in the serous glands in the mouth, which secrete part of the saliva, and is produced as a mammal eats. Lingual lipase has an optimum pH of 2.0-6.5 and is active in the absence of bile salts.⁹ Lingual lipase activity continues in the stomach after food is swallowed.¹⁰ A closely related enzyme is pregastric esterase (PGE), which has similar morphology, sequence, and activity to lingual lipase.¹¹ PGE is secreted in the glossoepiglotic area. As used herein, lingual lipase and pregastric esterase are essentially synonymous. Both are believed to play a much more important role in neonates and infant mammals than other lipases, including other preduodenal lipase variants.¹² ⁹ Table 3 in Hamosh 1994, see also Manson 1997, n. 4¹⁰ Sweet, B. J., et al. “Purification and characterization of pregastric esterase from calf,” Arch Biochem Biophys. 1984, 234, 144-150, https://doi.org/10.1016/0003-9861(84)90335-7; see also Manson 1997, n. 4¹¹ Table 3 in Hamosh 1994, n. 7¹² Id.

Lingual lipase/pregastric esterase are distinguished from other lipases, principally pancreatic lipase, that are delivered to the digestive tract after the food passes the duodenum. Biosynthetic (microbial, fungi and plant) sourced lipases may be viable alternatives to mammalian-sourced lingual lipase that may be utilized in some embodiments herein described.

In cases where a newborn cannot naturally suckle, infant malnutrition can be a significant problem due to fat maldigestion. Even newborns artificially fed naturally obtained colostrum frequently grow poorly, contributing to infant morbidity and mortality. This problem is a challenge with any mammal where breast feeding or natural suckling on the mother does not occur. This includes economically important farm animals, as well as humans, since many human mothers choose to not (or are unable to) breastfeed newborns. This problem is also significant in the dairy industry, where mothers may be removed from calves very soon after birth.

As used herein, “naturally suckle” means suckling from the mother's mammary glands, which include udders on cattle and goats, or breasts on humans. “Artificial suckling” or feeding means an infant fed by a bottle, artificial teat, via a tube, or fed solid food prematurely, that is, prior to a normal age for weaning.

It has been suggested that fats in colostrum, milk, or milk substitutes are not properly digested in infants in the absence of lingual lipase.¹³ The present inventors speculate that pregastric esterase (including lingual lipase) is not produced in artificially-fed infants, regardless of the quality of the milk, and that the lack of lingual or pregastric esterase lipase prevents an efficient breakdown of fats to fatty acids, so the artificially fed newborn is not digesting enough fat. ¹³ Hamosh M, Scanlon J W, Ganot D, Likel M, Scanlon K B, Hamosh P. “Fat Digestion in the Newborn: Characterization of lipase in gastric aspirates of premature and term infants,” J. Clinical Investigation 1981, 67(3):838-846, doi: 10.1172%2FJC1110101

The release of lingual lipase into the mouth in infant mammals is normally a direct result of a suckling action.¹⁴ The reason for the poor production of lingual lipase in some circumstances is unclear, since an infant may engage in a sucking action with both bottle-feeding and natural feeding. One possible explanation is that the milk or nipple from a bottle is the wrong temperature. Another explanation is that an infant usually spends substantial time with the mother for the first few days at least, suckling up to 10 times a day. This is more frequent than bottle fed infants (in bovines, calves are normally bottle fed twice per day for two weeks, then once per day until weaning), and each time the infant suckles, some lipase or pregastric esterase is released, so naturally suckled infants may get a larger dose of naturally produced lingual lipase or pregastric esterase. Another possible explanation is that a naturally sucking infant has to suck and prod the breast or udder to stimulate milk let down. So the naturally suckling infant will have lipase or pregastric esterase immediately available before the mother's milk has even arrived in its mouth. ¹⁴ Smith, L. J., Kaminsky, S. and D'souza, S. W. “Neonatal Fat Digestion and Lingual Lipase,” Acta Pædiatrica, 1986, 75 913-918. doi: 10.1111/j.1651-2227.1986.tb10316.x

Others have suggested lipase additives in animal feeds, for example in US2010/0239559, which discloses the use of lipases derived from plant or fungal sources (US2010/0239559 at ¶0045). See also US2017348198A1 and US2013/0280228. However, reports have emerged that lipase additives in animal feed were ineffective in providing measurable benefits. For example, Dierick and Decuypere reported that microbial lipase in pig diets showed no improvements in fat digestibility.¹⁵ Another report failed to detect increments of body weight gain, feed efficiency and energy digestibility with the use of an exogenous lipase in piglets.¹⁶ ¹⁵ Dierick N. A.; Decuypere, J. A., “Infuence of lipase and/or emulsier addition on the ileal and faecal nutrient digestibility in growing pigs fed diets containing 4% animal fat,” J. Sci. Food. Agric., 2004, 84(12) 1443-1450, https://doi.org/10.1002/jsfa.1794¹⁶ Officer, D. I., “Effect of multi-enzyme supplements on the growth performance of piglets during the pre- and post-weaning periods,” Animal Feed Sci Tech, 1995, 56(1-2), 55-65, https://doi.org/10.1016/0377-8401(95)00825-8

The foregoing discussion is not limited to infant or neonatal mammals. The principle of lipase deficiency is applicable to animals of any age with a lipase deficiency or fat maldigestion disorder.¹⁷ ¹⁷ See e.g., Sams L., Paume, J.; Giallo, J.; Carriere, F. “Relevant pH and lipase for in vitro models of gastric digestion,” Food Funct. 2016, 7, 30-45, DOI: 10.1039/c5fo00930h.

Lingual lipase of bovine source and other pregastric lipases are used industrially in the making of cheese.¹⁸ This disclosure provides for the combination of lipases from any source in combinations with unique biologically active components for mammalia feedings to address fat maldigestion. ¹⁸ http://blog.cheesemaking.com/about-lipase/(downloaded Jul. 3, 2018)

### SUMMARY OF THE INVENTION

To address the problem of improper digestion of fats in artificially fed infant mammals, a nutritional composition is provided for neonatal mammals who are not naturally suckling. In an embodiment, the composition includes a preduodenal lipase or esterase selected from lingual lipase, pregastric lipase, gastric lipase, and pregastric esterase, or a combination thereof, and butterfat, and a probiotic, and a prebiotic. The preduodenal lipases and esterases of this invention do not require a co-factor for lipase activity. In an embodiment, the preduodenal lipase is from a mammalian source or is a mammalian-derived protein produced biosynthetically. In an embodiment, the composition uses milk from the same species as the infant. In an embodiment, the composition uses milk from another species.

In an embodiment, the composition is used in a mammal of any age having a maldigestion disorder with an inability to digest fats properly.

The mammal may be human, or ruminant, a porcine, a horse, a camelid, a dog, a cat, or any mammal that suckle-feeds infants. Ruminants include a bovine, a buffalo, a deer, a goat, and a sheep.

In an embodiment, a nutritional composition is provided for neonatal mammals who are not naturally suckling comprising a combination of a pre-duodenal lipase or esterase selected from lingual lipase, pregastric lipase, gastric lipase, and pregastric esterase, or a combination thereof, butterfat, micellar casein, a probiotic and a prebiotic.

In an embodiment, nutritional composition is provided for neonatal mammals who are not naturally suckling comprising a combination of a pre-duodenal lipase or esterase selected from lingual lipase, pregastric lipase, gastric lipase, and pregastric esterase, or a combination thereof; butterfat; micellar casein; undenatured whey protein; a probiotic and a prebiotic.

In an embodiment, the nutritional composition may be based on mammalian milk or an artificial milk or milk replacer intended as a feed for infants or older mammals, wherein the lipases and or fat and other nutrients or nutrition factors are a component of the composition.

In an embodiment, a method is provided, using lipases from any source as a nutritional supplement to improve the digestion for infant mammals, stressed mammals, failure-to-thrive mammals, prematurity, post-surgical mammals and mammals with fat maldigestion. In an embodiment, the method comprises a nutritional supplement of lipases combined with any carrier/foodstuff including micronutrients, macronutrients such as milk solids, isolated soy proteins, micellar casein, carbohydrates, a prebiotic, a probiotic, non-denatured whey protein, bioactive dietary components such as omega 3 fatty acids, caffeine, plant anti-inflammatory components, or digestive aids such as other enzymes formulated for infant and older mammals.

In an embodiment, a method is provided for improving the digestion of triacylglycerol fats in vivo, in a mammal neonate, or older mammals, comprising lingual lipase from an animal source, or lipases from plant, or biosynthetic sources as a nutritional supplement in milk, a feeding formula for the neonate or a nutritional supplement.

### DETAILED DESCRIPTION

The present invention addresses the problem of malnutrition in newborn mammals, especially those that are not naturally fed at the breast or udder (in the case of bovines), those with failure-to-thrive, premature, nutritionally stressed, and more mature mammals with a fat maldigestion disorder. With regard to neonates, it is postulated by the present inventors that malnutrition can occur because of a lack of hydrolase activity in a newborn that is not naturally suckled. Accordingly, this invention provides compositions and methods containing lipases or esterases from mammalian sources that may address these issues. This invention is particularly applicable to infants in their first 48 hours post-partum, where nutritional deficiencies may first appear. However, the compositions and methods of this invention may also be of value in older infants or more mature mammals suffering from a fat maldigestion disorder.

A fat maldigestion disorder is any condition in which fats are not normally digested. The absorption of dietary fat is very efficient in healthy adults and as little as 4-5% of the ingested fat is excreted.¹⁹ By contrast, the process is much less efficient in newborns and especially in premature infants, where it has been measured (by fat in the feces) as low as 65% absorption,²⁰ i.e., at least 35% of dietary fat is not absorbed. It has also been reported that as little as 79% of fat in cystic fibrosis patients is absorbed.²¹ These all represent fat maldisgestive conditions in which the mammal is unable to effectively digest fats. The problem is particularly severe if it results in malnutrition. ¹⁹ Margit Hamosh, n. 1.²⁰ Id.²¹ Mini Kalivianakis, et al. “Fat malabsorption in cystic fibrosis patients receiving enzyme replacement therapy is due to impaired intestinal uptake of long-chain fatty acids”, Am. J. Clinical Nutrition, 1999 69(1) 127-134, doi: 10.1093/ajcn/69.1.127

Infant bovines born into the industrial farming model, especially dairy farming, are typically removed from the mother and the calving environment almost immediately after birth. Mis-mothering, calf-cow rejection, and adverse environmental or hygiene conditions mean that leaving the calf on the cow is often not effective or desirable. The cow joins the milking herd. Calves are raised separately from their mother, using milk or milk replacers. These infants frequently fail to thrive, meaning their weight gain, energy level, and overall health may be inferior to naturally-fed calves.

The present inventors have observed that raw milk, including colostrum, or milk replacers including reconstituted milk from milk powders, when fed to bovine newborns often makes the calf ill, resulting in a failure-to-thrive. This failure-to-thrive can be a significant economic problem. By contrast, calves that are sustained by the natural suckling off the mother thrive, compared to animal infants who are removed from their mother and reared using other less-natural systems. In modern dairy farming, bottle and tube feeding are employed with newborn calves that are taken away from the mother shortly after birth. These latter animals often fail to thrive.

Accordingly, the present inventors postulate that infants who do not, for whatever reason, naturally suckle at the udder or breast (i.e., the mothers' mammary glands) are susceptible to fat maldigestion disorders in the first few days after birth and may suffer from malnutrition, diarrhea, dehydration and needless mortality. Although the above discussion mentions bovines, infants from other species, including humans, may experience the same problems.

Bovine calves given lingual lipase with artificially fed milk (i.e., not at the mother's udder) are less sickly, more energetic, have shiny coats, sweet good smelling stools (indicating better digestion), grew faster and put on more weight in a fixed amount of time due to being able to digest milk more efficiently, and also consume more milk than those not fed naturally from the mother.

The lack of stimulation of the lingual glands on the tongue may deny the infant the necessary lipase to immediately modify the butterfat in milk by contact with the secreted lipase. Moreover, the presence of abundant free fatty acids (FFAs) creates an environment that is positive for desirable flora and negative to undesirable flora, i.e., it improves the health of the microbiome.

The proposed lack of lingual lipase or pregastric esterase exists in bottle fed calves, even though such calves may suckle on a bottle or other artificial teat. Since these devices are much more efficient at delivering milk than a natural teat, the feeding time is much shorter, which may account for the lack of lingual lipase. Moreover, many calves do not suckle well. Compromised calves (cold, premature, difficult birth, mis-mothered, abandoned, etc.) often have temporary neurological challenges, which compromises the suckling reflex and mechanisms, and may therefore reduce the stimulation of natural lingual lipase. Another issue is that in the dairy industry, some calves are bucket fed from day one, so they do not suckle at all. Yet another factor is that bottle fed system producers and extension services may recommend limited milk volumes as a cost and labor saver, and to mitigate nutritional scours (diarrhea) that commonly occur when calves are fed to appetite. Diarrhea and insufficient nutrition can lead to dehydration and death. This advice may underestimate how much milk calves can consume if their digestive systems are healthy. Another factor is that artificial systems usually have an environment that stresses the calf to some degree—different smells, tastes, touches (for example, the mother licking the calf) etc. This may diminish normal suckling effects and natural lipase production.

Another issue is that fat maldigestion results in malabsorption of nutrients. This problem may be addressed in the inventive formulations by the addition of nutrients, both macro and micro nutrients, in various forms and bioactive nutrient components, For example, a macro nutrient may be micellar casein or isolated soy protein or omega 3 fatty acids or a carbohydrate such as lactose or glucose, or a digestive aid including enzymes. For example, bioactive nutrition components may be caffeine, isoflavones, growth factors, anti-inflammatory components or anti-oxidant plant pigments.

### Lipases and Esterases

The present inventors postulate that a lack of essential preduodenal lipases, such as pregastric esterases or lipases (PGE's) in non-naturally fed infants deprives the infant of correct nutrition. The absence of PGE's in raw milk, pasteurized milk, or substitute milk formulae can result in infant ill-thrift or illness due to inefficient digestion. This may be caused by the known low activity of pancreatic and intestinal lipases in newborn mammals.²² Thus, in the absence of PGE, there may be insufficient lipase activity further down the digestive tract to break down the triglyceride fats in butterfat in milk. This same principle may apply in other fat maldigestion conditions not involving neonates or infants, such as cystic fibrosis, pancreatic lipase insufficiency, non-alcoholic fatty liver, alcoholic fatty liver or post-surgical conditions. In these situations, lipases may be useful as a component of a nutritional supplement to aid in fat digestion. Cystic fibrosis patients often have pancreatic insufficiency and fail to produce sufficient pancreatic lipase which can cause fat maldigestion. ²² Note 1, at 615, col. 2, third para.

The inventors have found that the addition of any of several esterases or lipases to infant formula for non-naturally suckling infants significantly improves the thrive factor in the infant, which presumably is a result of improved digestion of fats and development of a functional digestive microbiome.

In addition, the present inventors have found that the addition of lingual lipase to milk fed artificially to calves improves survival and reduces infant mortality. A potentially important element however is that different species have different lipases, and the present inventors believe the lipase must be matched to the species. Preferably, the lipase used is native to the species, for example bovine lingual lipase being added to feed for bovine calves. Similarly, human lingual lipase will perform better in humans. Conversely, microbial or plant lipases are unlikely to confer significant dietary benefits when fed to e.g., calves or humans, when required to work in situ.

Lingual lipase is a member of a family of digestive enzymes called triacylglycerol lipases, EC 3.1.1.3, that use the catalytic triad of aspartate, histidine, and serine to hydrolyze medium and long-chain triglycerides into partial glycerides and free fatty acids. The enzyme, released into the mouth along with the saliva, catalyzes the first reaction in the digestion of dietary lipid, with diglycerides being the primary reaction product. Lingual lipase has pH optimum pH of 4.5-5.4, and catalyzes the hydrolysis of esters in that absence of bile salts. The lipolytic activity continues in the stomach after food is swallowed, and it has been proposed that fats generally may not digest properly in neonates in the absence of lingual lipase.²³ Enzyme release is signaled by autonomic nervous system after ingestion, at which time the serous glands under the circumvallate and foliate lingual papillae on the surface of the tongue secrete lingual lipase to the grooves of the circumvallate and foliate papillae. ²³ Note 1

Other lipases, such as pancreatic lipases, lipases present in milk, and lipases from plant or fungal/biosynthetic sources, typically require a co-factor for the lipase activity, in particular, bile salts. No co-factor is required for animal-derived PGE's, including lingual lipase.²⁴ This is a potentially important feature for the lipases of this invention. ²⁴ Note 1 at 143, bottom.

The lipase for this invention may be obtained from mammalian sources. For example, lingual lipase used in cheese manufacture is obtained from tongues from calves, kids, lambs. Bovine and other mammalian lingual lipases are commercially available.

In an embodiment, the lipases or esterases for this invention may be mammalian enzymes produced synthetically, for example by inserted an appropriate DNA sequence into an expression system and cultivating the organism to produce the enzyme. Exemplary expression systems include bacteria such as E. coli and B. subtilis, and yeasts such as Saccharomyces. Many other expression systems are well known in the art for making heterologous peptides.²⁵ ²⁵ See, e.g, Joan Lin Cereghino James M. Cregg, “Heterologous protein expression in the methylotrophic yeast Pichia pastoris,” FEMS Microbiology Reviews, Volume 24, Issue 1, 1 Jan. 2000, Pages 45-66, https://doi.org/10.1111/j.1574-6976.2000.tb00532.x

The amino acid sequence of potential human and animal lingual lipase and pregastric esterases is known.²⁶ ²⁶ See for example “Cloning and expression of cDNA encoding human lysosomal acid lipase/cholesteryl ester hydrolase. Similarities to gastric and lingual lipases” J. Biol. Chem. 266 (33), 22479-22484 (1991). The sequence of bovine pregastric esterase is also known, (Timmermans, M. Y., Teuchy, H. and Kupers, L. P., The cDNA sequence encoding bovine pregastric esterase, Gene 147 (2), 259-262 (1994); NCBI NP_776528.

The lipases and esterases of value in this invention may be, but are not necessarily, species specific. That is, a lipase from one species, for example a bovine, may not be useful, or may have reduced efficiency, in hogs for example. In any event, the inventors believe that lipases from non-mammalian sources, such as plants or bacteria, which have been suggested previously as supplemental feeds, are unlikely to confer any significant benefit to humans or economically important animals when required to work in situ.

### Components of Natural Milk

In an embodiment, this invention discloses a feed or nutritional supplement product containing lingual lipase and or other lipases or esterases from mammalian sources that do not require bile acids for activation, for use with neonate or infant and older mammals. In an embodiment, the feed product is natural or artificial milk or a nutritional supplement. As used herein, “natural milk” is milk or colostrum from the same species as the neonate. As used herein “formulated natural milk” is milk that is not obtained from a lactating mother of the same species (for example, bovine milk fed to human infants). “Artificial milk” includes any liquid feed product or a manufactured formula that may be based on milk (for example, bovine milk) but has significant additional ingredients, or a manufactured formula not based on milk at all. In an embodiment, this invention discloses lingual lipase or lipases from mammalian sources as a nutritional supplement in natural or artificial milk fed artificially to neonatal mammals.

Casein is the primary protein in bovine milk. Approximately 80% of the proteins in bovine milk and between 20% and 45% of the proteins in human milk are casein. Casein is relatively hydrophobic, making it weakly soluble in water. It occurs natively in milk as a suspension of particles, called casein micelles (also termed herein “micellar casein”), which show a limited resemblance to surfactant-type micelles in the sense that the hydrophilic parts reside at the surface of the micelles and casein micelles are spherical. However, in contrast to surfactant micelles, the interior of a casein micelle is highly hydrated. The caseins in the micelles are held together by calcium ions and hydrophobic interactions. Any of several molecular models could account for the special conformation of casein in the micelles. Casein is a principal component of milk protein concentrate (MPC), which is commercially available and used as an additive in many food products.

During digestion, casein becomes truly insoluble from the action of chymosin, which cleaves off the kappa casein and destabilizes the micelle, allowing the modified micelle to have both negative and positive charges. The micelles rotate allowing positive to attach to negative charged regions of the modified micelles and a gel is formed in the stomach.

Whey protein is another macronutrient derived from milk with potent nutritive value. Preferably, non-denatured whey is used. Whey/immunoglobulin (antibody carrying) compounds are a component in MPC in non-denatured form, or it may be added, for example as sweet whey powder (which is non-denatured but may be pasteurized) to the inventive formulations.

In an embodiment, the feed product of this invention also includes lactose, the sugar in natural mammalian milk. The feed product of this invention ideally will contain about 2% to 7% by weight of lactose. Lactose may be added directly as lactose powder to the inventive formulations, or a component such as sweet whey or sweet cream may used, both of which contain lactose.

### Additional Ingredients

The inventive compositions and methods may include supplemental vitamins, minerals, or other nutrients. Supplemental nutrients may include, for example, kelp (a source of vitamins), additional vitamins or minerals (also termed “micronutrients”), macronutrients such as proteins, carbohydrate, and fats. Macronutrients for the inventive compositions and methods include isolated soy proteins, omega-3 fatty acids such as alpha-linolenic acid (ALA), docosahexaenoic acid (DHA) or eicosapentaenoic acid (EPA), or a combination thereof, lactose, a prebiotic, and a probiotic.

A potential issue with newborns is that no bacteria are present in the digestive tract of a newborn animal, which can cause digestive problems. Colostrum can be collected from mothers mechanically and then fed to calves artificially by bottles or a feeding tube down the throat directly into the stomach. But in the absence of appropriate flora in the gut, the colostrum may not digest properly. This problem may be addressed in the inventive formulations by the addition of a probiotic with or without a prebiotic.

A probiotic adds beneficial digestive bacteria, which are an additional requirement for nutrition. Prebiotics are food ingredients that induce the growth or activity of beneficial microorganisms in the gut.²⁷ Prebiotics can alter the composition of organisms in the gut microbiome. The addition of prebiotics and probiotics can populate the gut with appropriate bacteria that are required for digestion. ²⁷ Hutkins R W et al., “Prebiotics: why definitions matter” Curr Opin Biotechnol. 2016 February; 37:1-7. doi: 10.1016/j.copbio.2015.09.001. Epub 2015 Sep. 29.

Prebiotics stimulate the growth or activity of advantageous bacteria that colonize the large bowel by acting as substrate for them. In an embodiment, a prebiotic may be a composition of inulin, fructo-oligosaccharides (FOS), galactooligosaccharides (GalOS), lactulose, or pectin.²⁸ Inulin is a polysaccharide composed mainly of fructose units (fructans), and typically has a terminal glucose. It consists of chain-terminating glucosyl moieties and a repetitive fructosyl moiety, which are linked by β(2,1) bonds. Because of the β(2,1) linkages, inulin is indigestible by the human enzymes ptyalin and amylase, which are adapted to digest starch. As a result, it passes through the upper digestive tract intact. Only in the colon do bacteria metabolize inulin contributing to its functional properties: reduced calorie value, dietary fiber, and prebiotic effects. Without color and odor, it has little impact on sensory characteristics of food products. After reaching the large intestine, inulin is converted by colonic bacteria to a prebiotic gel that is highly nourishing to gut microflora. Sources of inulin include bananas, chicory root, and Jerusalem Artichoke (a tuber vegetable native to North America). ²⁸ Belén Gómez, et al., “Purification, Characterization, and Prebiotic Properties of Pectic Oligosaccharides from Orange Peel Wastes,” J. Ag. Food Chem., 2014 62 (40), 9769-9782 DOI: 10.1021/jf503475b

Significantly, the enzymes mentioned above, ptyalin and amylase, are presumably not limited to humans. In particular, immature ruminant animals such as bovines or goats are not actually ruminants with a multichamber stomach at birth. Bovines and goats have a monogastric digestive tract at birth until age 6-12 weeks, and it is believed that a prebiotic such as inulin will pass through the digestive tract of young ruminants to lodge in the large intestine and exert a prebiotic effect before these young animals develop a true ruminant upper digestive tract.

### Compositions

In an embodiment, the inventive methods and compositions includes lipases or esterases, and may include ingredients such as one or more of milk (dried or fresh), kelp, additional vitamins or minerals, macronutrients such as micellar or native casein, isolated soy proteins, omega-3 fatty acids, carbohydrates including lactose, a prebiotic, and a probiotic. In an embodiment, for example, the milk is a carrier, and the kelp is a source of minerals and vitamins beneficial to the newborn.

The amount of lipases in the feed supplement can be adjusted based on the amount of fat in natural milk for the species or the amount of fat in the diet or nutritional supplement as appropriate for the mammalian species. For example, domestic cattle milk has about 4-5% butterfat, buffalo milk has 7-9% butterfat in natural milk, domestic pig milk has about 7-8% fat, and human milk has about 4.5% fat. For example, additional lipase would be added to a high fat content natural milk like that from a buffalo. Diets for mammals with fat maldigestion, failure-to-thrive, nutritional stress and post surgical conditions will vary according to recommended nutrition protocols for each species, varying ages, conditions and sizes. Thus lipases and additional components and amounts of each will vary.

In an embodiment, the feed product of this invention may include added butterfat. Butterfat may be a superior form of fat for newborns, especially bovines. Normally, the butterfat of this embodiment is from whole milk, and the butterfat is the natural fat (cream) present in natural milk.

The inventive compositions and methods are expected to be equally valid for other mammals besides bovines, including other economically important farm or domestic animals, such as other ruminants such as buffalo, deer, goats, sheep, porcines (hogs), horses, camelids (camels, llamas, or alpacas), and domestic pets such as dogs, or cats. For example, in species such as porcines that produce large litters, one or two infants may be excluded and left to die naturally. This invention allows these potentially valuable animals to be rescued and raised to maturity.

This invention may also be of value to exotic and endangered animals such as those raised in zoos or nature reserves. Newborns are often removed from mother at birth or shortly thereafter in these environments to enhance survival prospects.

This inventive compositions and methods may also be of value for humans, and used in human infant formulas and in humans with fat maldigestion. Current guidelines suggest that humans should be fed at the breast exclusively for the first six months of life,²⁹ yet many mothers are unable or unwilling to do that. Even where there is not obvious malnutrition, the addition of lipases to infant formula or natural human milk may improve digestion and infant health in neonates. The addition of lipase containing nutritional supplements formulated with nutrients and bioactive nutritional components to diets for specific malnutrition conditions in humans may improve health. ²⁹ World Health Organization, “Infant and young child feeding” Fact sheet, http://www.who.int/en/news-room/fact-sheets/detail/infant-and-young-child-feeding (downloaded Jul. 4, 2018)

### EMBODIMENTS

A feed of this invention provides a nutritional supplement added to natural milk or a milk replacer or formulated into a non-milk nutritional supplement. In an embodiment, the supplement may include a range of ingredients depending on the species, weight, age or health condition. An example for calves may be:
Lipase powder 0.001 to 10 grams
Prebiotic 0.001 to 10 grams
Probiotic 0.001 to 10 grams
Dried powdered kelp 0.001 to 10 grams
Mineral mix 0.001 to 3 grams
Powdered Non-fat dried milk 1.0-20 grams

These ingredients would be blended into sufficient natural milk or a milk substitute so that there is approximately 5 g of this supplement in 4 liters (about one US gallon).

In an alternative embodiment for a calf feed, the ingredients may be:
Lipase powder 0.001 to 10 grams
Prebiotic 0.001 to 10 grams
Probiotic 0.001 to 10 grams
Dried powdered kelp 0.001 to 10 grams
Mineral mix 0.001 to 3 grams

This would be diluted to about 4-5 g of solid in 4 liters of milk or a milk substitute.

In a more specific embodiment for bovine calves, the following materials may be used:
Lipase powder 1.0 gram
Prebiotic 3.0 grams
Probiotic 4.5 grams
Dried powdered kelp 3.0 grams
Mineral mix 3.0 grams
Powdered Non-fat dried milk 1.0 grams
This mixture is diluted in 16 liters of natural milk or a milk substitute.

### EXAMPLES

### Example 1

A supplement for calves was prepared with the following ingredients:
Lipase powder 1.0 gram
Prebiotic 3.0 grams
Probiotic 4.5 grams
Dried powdered kelp 3.0 grams
Mineral mix 3.0 grams
Powdered Non-fat dried milk 1.0 grams
All ingredients are commercially available in the USA.

This mixture was blended into 4 US gallons (15.1 liters) of natural cows milk.

### Evidence of Efficacy

A Missouri dairy farm with about 850 milking cows used this lingual lipase supplement as part of their calf rearing operation. Over a thirty-month period, the supplemented milk was given to more than 1,600 heifer calves, along with more than 400 male calves. The addition of the lingual lipase supplement was continued for up to 5 weeks, or in the case of the retained male calves, up until they were sold at 1-4 weeks of age. Animals were allowed to suckle vigorously on artificial “calfetaria” teats, including continued suckling for 4-5 minutes after the provided milk was consumed. Performance of the calves improved subsequent to the inclusion of lipase. The calf rearing system, the management, and the feed and feeding systems remained the same as previous years without the lingual lipase supplement. Changes measured or observed included:
improved digestion as evidenced by stool condition and consistency;
zero nutritional scours;
improved intelligence as evidenced by improved vigor, mobility, and herd instincts;
stronger appetite;
improved immunity;
improved and accelerated growth rate;
improved performance as juveniles and adults
Lower mortality;
increased survival rates of calves compromised by factors such as difficult birth, abandonment by their mother, extreme cold or heat stress;

The improved start to life enabled by the inclusion of lingual lipase translated to accelerated growth and vigor and enabled these calves to thrive and show better weight gain through to weaning weights.

### Example 2

A commercial Missouri deer farm calving 80-90 hinds each spring added the same lipase supplement from Example 1, in the milk or milk substitutes given to orphaned or mis-mothered fawns. Mortality of these fawns dropped from over 50% average for the previous four seasons, to less than 5% for the two years that lipase was added. No other factors changed during that time.

### Example 3

This is an adjunct formula for supplemental feeding of animals also receiving whole milk. There is no fat in this formula.

| Material | Amount % (w/w) |
|---|---|
| NFDM | 80.00 |
| Lipase (bovine source) | 13.33 |
| Chicory Flour (prebiotic) | 6.67 |
| Total | 100.00 |

NFDM = nonfat dry milk.

For bovine calves, the lipase is of bovine origin, which is commercially available.

For use, 13.33 g of this formula is added to 26 gals (98 L) of milk (natural or artificial).

### Example 4

This is a complete milk replacer formula.

| Material | Amount % (w/w) |
|---|---|
| MPC 70 | 17.06 |
| Sweet Whey | 24.53 |
| Lipase (bovine source) | 7.76 |
| Chicory Flour (prebiotic) | 3.88 |
| Sweet Cream | 46.76 |
| Total | 100.00 |

MPC = milk protein concentrate 70% (commercially available)

For bovine calves, the lipase is of bovine origin, which is commercially available. The sweet whey in this experiment was not denatured.

This formula is reconstituted by adding 100 g to 900 mL of water to make 1 L of formula.

1. A nutritional composition for a mammal comprising a combination of:
a. a preduodenal lipase or esterase that does not require a co-factor for activity, selected from lingual lipase, pregastric lipase, gastric lipase, and pregastric esterase, or a combination thereof, wherein the preduodenal lipase or esterase is from a mammalian source or is a mammalian-derived protein produced biosynthetically;
b. butterfat; and
c. a probiotic and a prebiotic.

2. A nutritional composition comprising a combination of:
a. a preduodenal lipase or esterase that does not require a co-factor for activity, selected from lingual lipase, pregastric lipase, gastric lipase, and pregastric esterase, or a combination thereof, wherein the preduodenal lipase or esterase is from a mammalian source or is a mammalian-derived protein produced biosynthetically;
b. butterfat; and
c. a probiotic and a prebiotic,
wherein the composition is administered to neonatal mammals or a mammal with fat maldigestion.

3-5. (canceled)

6. The nutritional composition of claim 1 wherein the mammal is a ruminant, a porcine, a horse, a camelid, a dog, a cat, or a human.

7. The nutritional composition of claim 1 wherein the mammal is a ruminant selected from a bovine, a buffalo, a deer, a goat, and a sheep.

8. The nutritional composition of claim 1 wherein the mammal is less than 48 hours old.

9. The nutritional composition of claim 1 wherein the mammal is suffering from an inability to effectively digest fats.

10. The nutritional composition of claim 1 wherein the mammal is suffering from malnutrition from an inability to effectively digest fats.

11. The nutritional composition of claim 1, wherein the composition further comprises milk from the same species as the mammal or a different species of mammal.

12. The nutritional composition of claim 1, wherein the composition further comprises an artificial milk.

13. The nutritional composition of claim 1, wherein the composition further comprises an additive selected from one or more of kelp, omega-3 fatty acids,

14. The composition of claim 1, wherein the preduodenal lipase or esterase does not require a co-factor for activity, and the preduodenal lipase or esterase is selected from lingual lipase, pregastric lipase, gastric lipase, and pregastric esterase, or a combination thereof, wherein the preduodenal lipase or esterase is from a mammalian source or is a mammalian-derived protein produced biosynthetically, and butterfat, a probiotic and a prebiotic in the manufacture of a liquid composition for the treatment of fat maldigestion in a mammal.

15. A nutritional composition for neonatal mammals or mammals with fat maldigestion comprising a combination of:
a. a preduodenal lipase or esterase that does not require a co-factor for activity, selected from lingual lipase, pregastric lipase, gastric lipase, and pregastric esterase, or a combination thereof wherein the preduodenal lipase or esterase is from a mammalian source or is a mammalian-derived protein produced biosynthetically;
b. butterfat;
c. a probiotic and a prebiotic; and
d. micellar casein or non-denatured whey protein or both.

16. The composition of claim 15 further comprising lactose.

17. The composition of claim 15 further comprising a vitamin supplement, a mineral supplement, or an omega-3 fatty acid supplement, or a combination thereof.

18. A complete milk replacement for neonatal mammals or mammals with fat maldigestion comprising the composition of claim 15.

19-21. (canceled)

21. A nutritional supplement that is added to milk or a milk substitute for neonatal mammals or mammals with fat maldigestion comprising the composition of claim 1.

22. (canceled)

23. (canceled)
